(19) United States
(12) Patent Application Publication
(10) Pub. No.: US 2023/0203107 A1
(43) Pub. Date: Jun. 29, 2023

(54) PEPTIDE FOR TREATING SEPSIS DERIVED FROM RV3364C PROTEIN OF MYCOBACTERIUM TUBERCULOSIS

(71) Applicant: INDUSTRY-UNIVERSITY COOPERATION FOUNDATION HANYANG UNIVERSITY ERICA CAMPUS, GYEONGGI-DO (KR)

(72) Inventors: Chul Su YANG, Gyeonggi-do (KR); Da Eun LEE, Gyeonggi-do (KR)

(21) Appl. No.: 18/053,965

(22) Filed: Nov. 9, 2022

(30) Foreign Application Priority Data
Nov. 11, 2021 (KR) 10-2021-0155093

(51) Int. Cl. C07K 14/35; A61P 31/04; A61P 37/06

(52) U.S. Cl. CPC C07K 14/35; A61P 31/04; A61P 37/06; A61K 38/00

(57) ABSTRACT

Provided is an Rv3364c-derived peptide capable of directly binding to a BAR domain of SNX9. The Rv3364c-derived peptide of the present disclosure may regulate a TLR4-mediated inflammatory response depending on SNX9, and thus can be effectively applied to not only the use for the prevention and treatment of Mycobacterium tuberculosis infectious disease, but also the use for the prevention and treatment of sepsis and sepsis shock.

### CROSS-REFERENCE TO RELATED APPLICATION

This application claims priority to Korean Patent Application No. 10-2021-0155093 filed Nov. 11, 2021, the entire disclosure of which is incorporated herein by reference.

### SEQUENCE LISTING

The instant application contains a Sequence Listing which has been submitted in .xml format and is hereby incorporated by reference in its entirety. Said .xml file was created on Nov. 9, 2022, is named “FPC-2022-0205US SEQ.xml”, and is 33,277 bytes in size.

### BACKGROUND

### 1. Field of the Invention

One or more example embodiments relate to a peptide derived from an Rv3364c protein of Mycobacterium tuberculosis, and more particularly, to the use of a peptide derived from Rv3364c required for interaction with SNX9 for treating sepsis.

### 2. Description of the Related Art

Bacterial antigens cause an interaction between a host immune defense and a mechanism that allows bacteria to escape from the host immunity or to protect themselves. This host-pathogen interaction is very complicated by intracellular pathogens, such as Mycobacterium tuberculosis (MTB), a cause of tuberculosis. Rv3364c, known as a serine protease inhibitor of Mycobacterium tuberculosis, is an antigen which is expressed at a high level in macrophages exposed to Mycobacterium tuberculosis, and strongly expressed in culture supernatants and lysates of Mycobacterium tuberculosis and macrophages. Rv3364c contains a Roadblock/LC7 domain and is associated with outer/inner flagellar dynein of eukaryote and cytoplasmic dynein of Myxoccus xanthus, and serves to regulate the structure and function of each dynein. In addition, an Rv3364c effector protein binds to serine protease cathepsin G in the cell membrane of macrophages to inhibit its enzymatic activity and caspase-1 dependent apoptosis in its lower reaction pathway. However, the understanding of the interaction between Rv3364c and the host cell is still insufficient, and the understanding of the interaction between macrophages and Rv3364c is expected to make a significant contribution to the establishment of effective infectious disease treatment strategies.

Sepsis is defined as life-related organ dysfunction caused when a host response to infection is not regulated. When suffering from sepsis, an immune response initiated by the pathogen does not maintain homeostasis, and excessive inflammation and bacterial proliferation cause persistent pathological syndromes. The mortality rate of sepsis is close to 25%, and due to high incidence and mortality, global expenditure thereof is significant.

Although the level of understanding of the pathogenesis of sepsis has increased, a target treatment method is still insufficient.

Meanwhile, peptides and proteins have great potential as therapeutic agents. Currently, small molecule drugs of small sizes occupy most of the pharmaceutical market, but compared to these typical small molecule drugs, peptides and proteins have target specificity to have less side effects and toxicity, and have an advantage of being easily optimized using non-natural amino acids. Although peptides and proteins for therapeutic purposes are continuously being studied, methods for increasing systemic stability and delivery to specific sites have been discussed. In addition, the lack of target specificity of cell-penetrating peptides remains as a major obstacle in clinical development.

### LITERATURE OF RELATED ART

### Non-Patent Literature

EMBO Molecular Medicine 2020, vol.12, issue12: e12497 “Mycobacterium tuberculosis Rv2626c-derived peptide as a therapeutic agent for sepsis”

### SUMMARY

Example embodiments provide the use of a peptide including the amino acid sequence of a region involved in interaction with SNX9 in Rv3364c for treating sepsis.

According to an aspect, there is provided a pharmaceutical composition for preventing or treating sepsis including the peptide.

Additional aspects of example embodiments will be set forth in part in the description which follows and, in part, will be apparent from the description, or may be learned by practice of the disclosure.

In order to solve the above problems, the present disclosure provides a peptide derived from Rv3364c as a peptide for treating sepsis.

In the present disclosure, the Rv3364c-derived peptide is a peptide including the amino acid sequence of the region involved in the interaction with SNX9 in Rv3364c, and the minimum range of the region involved in the interaction with SNX9 is the 12-17 a.a amino acid sequence of the Rv3364c protein (SEQ ID NO: 14).

As one example embodiment of the present disclosure, the peptide for the treatment of sepsis may include one or two or more additional amino acids at the N-terminus and/or C-terminus if it does not affect the functional properties of the 12th and 17th amino residues (W₁₂ and F₁₇), and in a corresponding sense, the Rv3364c-derived peptide provided for the sepsis treatment use may include or consist of the amino acid sequence of SEQ ID NO: 2.

As another example embodiment of the present disclosure, the peptide for the treatment of sepsis may be a cell-penetrating peptide linked to the N-terminus of the Rv3364c-derived peptide, and the cell-penetrating peptide may be selected from the group consisting of HIV-TAT (SEQ ID NO: 15), TAT (SEQ ID NO: 16), dNP2 (SEQ ID NO: 17), VP22 (SEQ ID NO: 18), MPG (SEQ ID NO: 19), PEP-1 (SEQ ID NO: 20), EB1 (SEQ ID NO: 21), transportan (SEQ ID NO: 22), p-Antp (SEQ ID NO: 23), hCT(18-32) (SEQ ID NO: 24), KLA (SEQ ID NO: 25), and oligoarginine (SEQ ID NO: 26).

In addition, the present disclosure provides a pharmaceutical composition for preventing or treating sepsis including the peptide as an active ingredient.

In addition, the present disclosure provides a method for preventing or treating sepsis including administering the peptide to a subject.

The present disclosure also provides the use of the peptide for the manufacture of a medicament for the prevention or treatment of sepsis.

According to example embodiments, the present inventors confirmed that an Rv3364c protein derived from Mycobacterium tuberculosis reacted with SNX9 of a host, identified a region directly interacting with SNX9, and prepared an Rv3364c-derived peptide capable of directly binding to a BAR domain of SNX9. The Rv3364c-derived peptide may regulate a

TLR4-mediated inflammatory response depending on SNX9, and thus can be effectively applied to not only the use for the prevention and treatment of Mycobacterium tuberculosis infectious disease, but also the use for the prevention and treatment of sepsis and sepsis shock.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIGS. 1A and 1B illustrate results of screening candidate proteins predicted to potentially interact with rRv3364c in a CDI HuProt™ Human Proteome Microarray. Each figure illustrates a section of a protein array showing a positive response. A signal at 532 nm was stronger in an rRv3364c group than in a control group;

FIG. 2 illustrates a result of confirming an interacting protein by treating BMDM cells and HEK293T cells with rRv3364c and performing co-immunoprecipitation in order to screen proteins interacting with rRv3364c in vivo. Specifically, (A) illustrates a result of harvesting BMDM cells with different treatment times of 1 μg/ml rRv3364c, performing immunoprecipitation (IP) with αHis, and performing immunoblot (IB) using αSNX9, αYEATS4, αZBTB46, and αEGFLAM. WCL was used with αHis or αActin for the IB. (B) illustrates a result of co-transfecting 293T cells with Flag-Rv3364c and Myc-tagged SNX9, YEATS4, ZBTB46, or αEGFLAM, performing IP with αFlag (left) or αMyc (right), and then performing IB using αMyc or αFlag. WCL was used together with αHis or αActin for the IB;

FIGS. 3A and 3B illustrate results of confirming locations of rRv3364c in cells;

FIG. 3A illustrates a result of fractioning Raw264.7 into a nucleus and a cytoplasm with different treatment times of 1 μg/ml rRv3364c, performing IP with αHis, and then performing IB with αSNX9 or αYEATS4. WCLs were used together with αSNX9, αYEATS4, or αHis for the IB;

FIG. 3B illustrates a result of performing immunofluorescence using His-rRv3364c (Alexa Fluor 568) and SNX9 or YEATS4 (Alexa Fluor 488) antibodies in BMDM cells with different treatment times of 1 μg/ml rRv3364c. DAPI (blue) stains the nucleus, and a scale bar is 10 μm;

FIGS. 4 and 5 illustrate results of confirming regions where Rv3364c interacts with SNX9 and YEATS4;

FIG. 4 illustrates a result of confirming a region interacting with each protein in Rv3364c by designing an Rv3364c-derived peptide of a length of 10 a.a, preparing a Tat-rRv3364c peptide fused with a Tat peptide at an N-terminus, and then confirming the degree to which each Tat-rRv3364c peptide interferes with the binding of rRv3364c to SNX9 (A) and YEATS4 (C). Specifically, the result is a result of treating 293 cells transfected with Myc-SNX9 together with Flag-Rv3364c with a Tat-Rv3364c peptide (5 μM) for 12 hours, performing IP with αFlag, and then performing IB with αMyc. WCLs were used together with αMyc, αFlag, and αActin for the IB. In addition, immunoprecipitation and immunoblotting were performed on cells with different concentrations of a Tat-Rv3364c peptide highly reactive with a target (B and D);

FIG. 5 illustrates a result of searching for a region of an rRv3364c protein required for interaction with SNX9;

FIG. 6 illustrates a result of searching for a region of SNX9 that interacts with an rRv3364c protein. Specifically, the result is a result of designing a peptide isolating each domain of wild-type SNX9 (A), co-transfecting 239T cells with an Myc-SNX9 peptide together with Flag-rRv3364c, and then performing IP with αFlag (B) or αMyc (C) and performing IB with αMyc (B) or αFlag (C);

FIG. 7 is a schematic diagram illustrating regions where Rv3364c interacts with SNX9 and YEATS4 and locations in a cell;

FIGS. 8A to 8D illustrate results of confirming that Rv3364c interacts with SNX9 in macrophages to regulate a TRL4-mediated inflammatory response;

Specifically, FIG. 8A illustrates a result of treating 1 μg/ml rRv3364c or rVector in DMBM cells of WT, TLR2^{-/-}, TLR4^{-/-}, MyD88^{-/-}, TRIF^{-/-}, IRAK1^{-/-}, TRAF6^{-/-}, and TBK1^{-/-} mice for 18 hours, harvesting a culture supernatant, and then measuring the levels of TNF-α, IL-6, IL-12p40, and IL-10 through ELISA;

FIG. 8B illustrates a result of treating 1 μg/ml rRv3364c or rVector in DMBM cells of SNX9^{-/-} mice for 18 hours, harvesting a culture supernatant, and then measuring the levels of TNF-α, IL-6, IL-12p40, and IL-10 through ELISA;

FIG. 8C illustrates a result of pre-treating a TAT-Rv3364c peptide (₁₂WLVSKF₁₇) (1, 5, and 10 μM) in BMDM cells of SNX9^{+/+} or SNX9^{-/-} mice for 1 hour, stimulating the cells with 100 ng/ml LPS for 18 hours, harvesting a culture supernatant, and then confirming the expression level of cytokines through ELISA, and FIG. 8D illustrates a result of collecting the cells and confirming the phosphorylated forms of AKT, MAPK (ERK, p38, JNK), and IκBα and total IκBα through IB;

FIG. 9 illustrates a result of confirming that the interaction between SNX9 and p47phox and a Tat-rRv3364c (12-17 a.a) peptide inhibit ROS production in endosomes under LPS stimulation. Specifically, (A) illustrates a result (top) of pre-treating BMDM and THP-1 with a TAT-Rv3364c peptide (₁₂WLVSKF₁₇) (1, 5, and 10 μM) for 1 hour, stimulating the cells with 100 ng/ml LPS for 30 minutes, performing IP with αSNX9, and then performing IB with αp47phox and a result (bottom) of confirming the activity of NADPH oxidase. (B) illustrates a result of pre-treating BMDM with a TAT-Rv3364c peptide (₁₂WLVSKF₁₇) (1, 5, and 10 μM) for 1 hour, stimulating the cells with 100 ng/ml LPS for 30 minutes, performing IP with αSNX9, and then performing IB with αp22phox, αp67phox, and αp91phox. WCL was used together with αp22phox, αp67phox, αp91phox, or αActin in the IB;

FIGS. 10A-10C illustrate a result of tracking the movement of SNX9 in endosomes and observing signaling and molecular dynamics in endosomes by centrifuging and purifying sections of endosomes by sucrose flotation gradient assay and performing immunofluorescence microscopy;

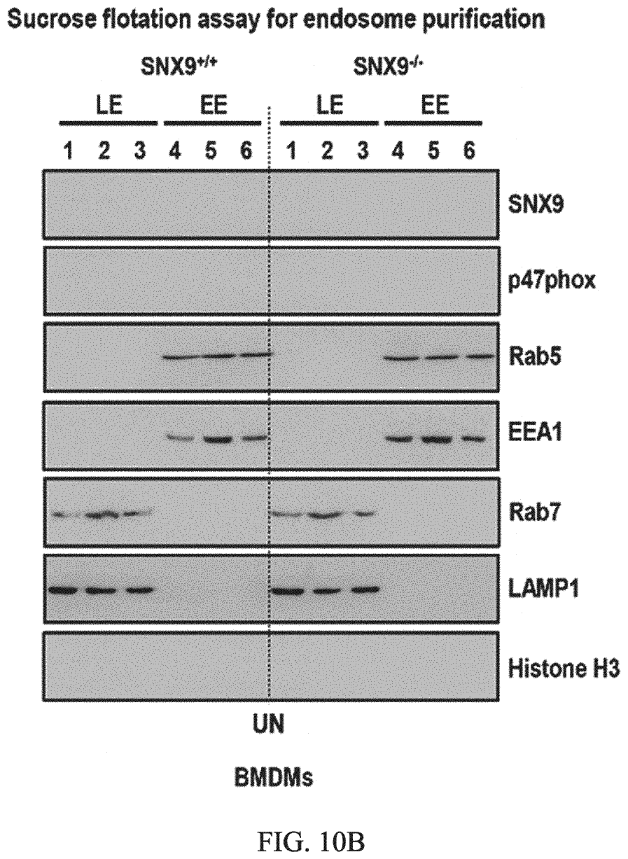

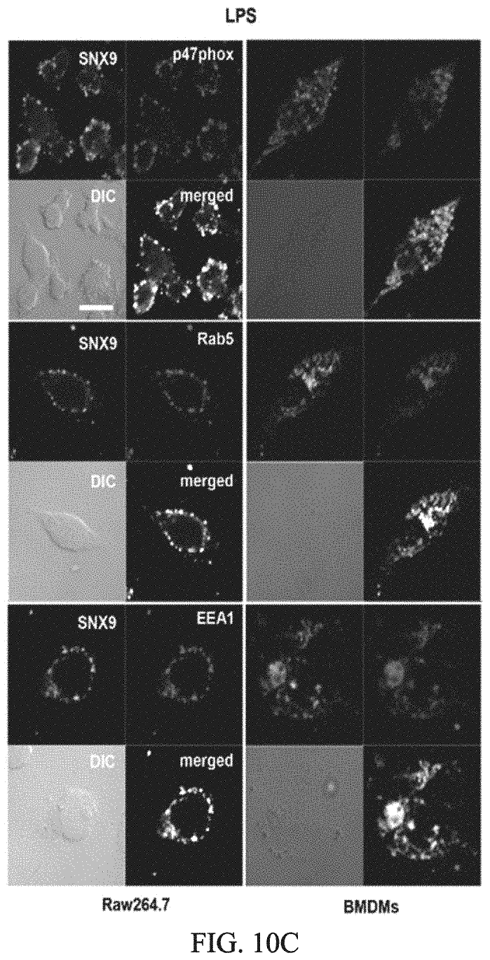

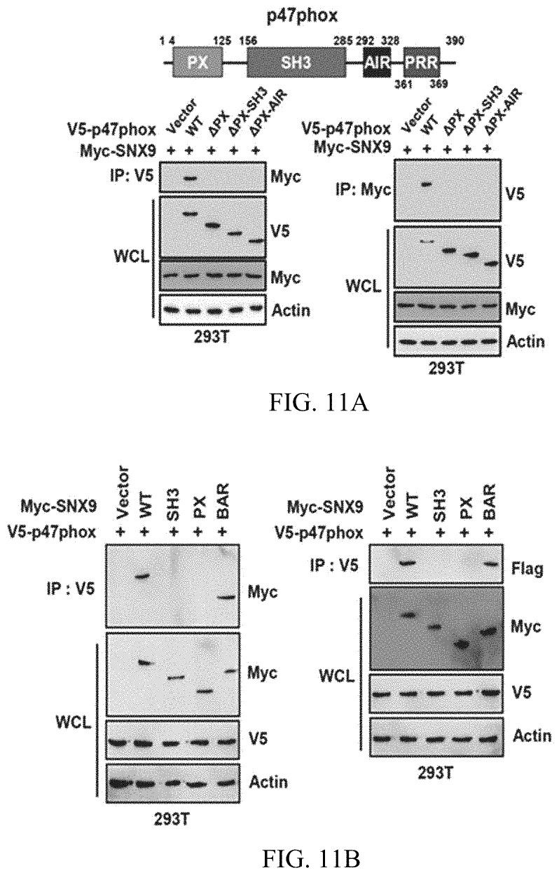

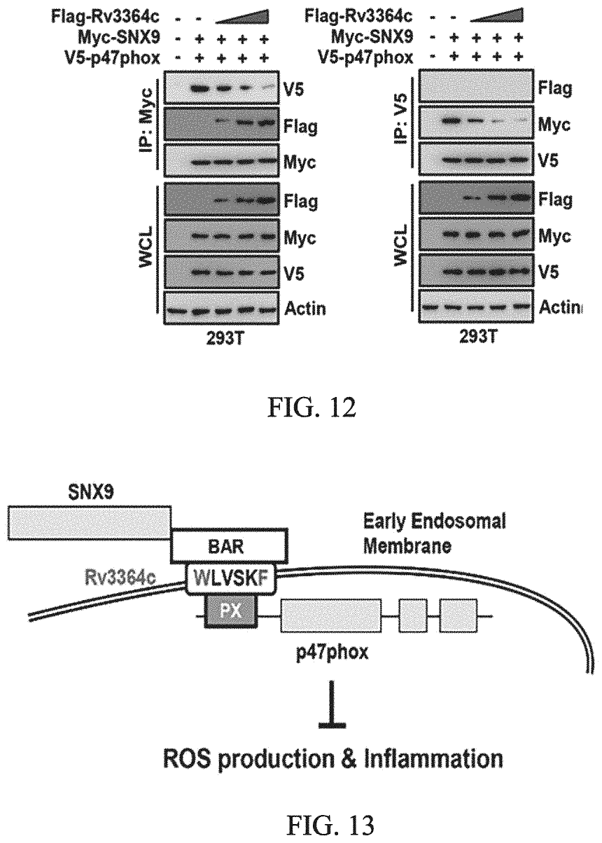

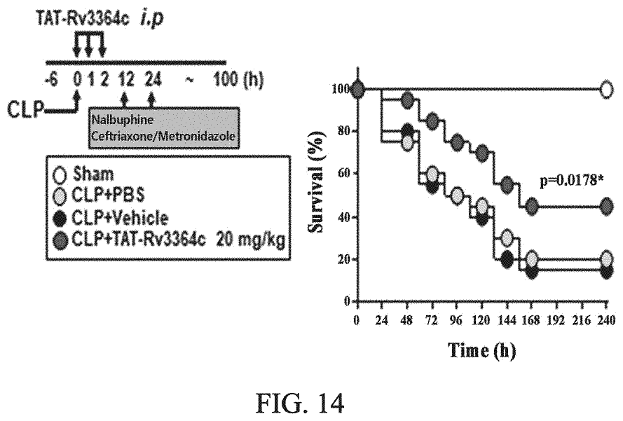

Specifically, the top of FIG. 10A illustrates a result of isolating the same volume fraction of BMDM of SNX9^{+/+} or SNX9^{-/-} mice using endosomal sucrose flotation gradient assay by SDS-PAGE in each gradient compartment, and examined through IB: Rab5 and EEA1 (EE marker), Rab? (LE marker), LAMP1 (LE and lysosomal markers), Histone H3 (nuclear marker). The bottom of FIG. 10A illustrates a result of immunostaining Raw264.7 or BMDM pretreated with a Tat-Rv3364c peptide (₁₂WLVSKF₁₇) (5 μM) for 1 hour and stimulated with 100 ng/ml LPS for 30 minutes through an antibody against SNX9 (Alexa Fluor 488) or p47phox (Alexa Fluor 568). The scale bar in the figure indicates 10 μm;

FIG. 10B illustrates a result of analyzing an endosomal membrane distribution using a sucrose flotation gradient in BMDM of SNX9^{+/+} or SNX9^{-/-} mice, in which the same volume fraction was isolated by SDS-PAGE in each gradient compartment and examined through IB: Rab5 and EEA1 (EE marker), Rab? (LE marker), LAMP1 (LE and lysosomal markers), Histone H3 (nuclear marker);

FIG. 10C illustrates a result of immunostaining Raw264.7 or BMDM pretreated with a TAT-Rv3364c peptide (₁₂WLVSKF₁₇) (5 μM) for 1 hour and stimulated with 100 ng/ml LPS for 30 minutes through an antibody against SNX9 (Alexa Fluor 488) or p47phox (Alexa Fluor 568). The scale bar in the figure indicates 10 μm;

FIG. 11A is a schematic diagram (top) of a p47phox domain structure and a result of co-transfecting p47phox and each domain of Myc-SNX9 and p47phox into 293T cells, performing IP with αV5 or αMyc, and then performing IB with αMyc, αV5, or αactin. FIG. 11B illustrates a result of co-transfecting p47phox and each domain of Myc-SNX9 and p47phox into 293T cells, performing IP with αV5 or αMyc, and then performing IB with αMyc, αV5, or αactin;

FIG. 12 illustrates a result of co-transfecting 293T cells with V5-p47phox and Myc-SNX9 together with Flag-Rv3364c (1, 5, 10 μg) for 12 hours, performing IP with αMyc (left) or αV5 (right), and then performing IB with αMyc, αV5, αFlag, or αactin; and

FIG. 13 is a schematic diagram of the regulation of a TLR signaling pathway mediated by rRv3364c. The Rv3364c-derived peptide ₁₂WLVSKF₁₇ directly interacts with a BAR domain of SNX9 to inhibit the interaction between SNX9 and p47phox in an early endosome induced under LPS stimulation, thereby reducing ROS production and the expression level of inflammatory cytokines.

FIG. 14 is a survival rate of CLP-induced sepsis mice administered with the Rv3364c-derived peptide.

### DETAILED DESCRIPTION

The present inventors found an effector protein of Mycobacterium tuberculosis that interfered with the action of a defense mechanism of a host, and confirmed a molecular action of a Mycobacterium tuberculosis-derived protein in a host cell. For example, it was confirmed that Rv2626c inhibited a TLR4 immune response by directly binding to a RING domain (N-terminus) of TNF Receptor-Associated Factor 6 (TRAF6) to prevent lysine 63-ubiquitination of TRAF6. A recombinant Rv2626c-CA peptide showed a significant therapeutic effect in a CLP-induced sepsis mouse model. In addition, it was confirmed that MPT63, a secreted protein of Mycobacterium tuberculosis, interacted with TBK1 and p47phox, and MPT64 interacted with TBK1 and HK2, and it was confirmed that a recombinant MPT protein was prepared by combining an interaction motif of each secreted protein to promote the reduction of the number of Mycobacterium tuberculosis in vitro and in vivo in macrophages.

According to continuous prior studies, the present disclosure is to provide the use of an Rv3364c-derived peptide for treating sepsis by confirming the interaction with molecules in a host cell of the Rv3364c protein derived from Mycobacterium tuberculosis, determining a mechanism of interference with a defense mechanism action of the host, and using the mechanism in reverse.

First, the present inventors confirmed whether Rv3364c interacted with proteins in a cell-free system through a customized protein binding assay in order to identify interacting factors in the host cell, selected 118 candidate proteins predicted to interact with Rv3364c, and confirmed the affinity of each candidate protein and Rv3364c to select a total of 4 types of proteins of SNX9 (sorting nexin 9), YEATS4 (YEATS domain-containing protein 4), ZBTB46 (zinc finger and BTB domain-containing 46), and EGFLAM (EGF-like Fibronectin type III and Laminin G domains). Subsequently, in order to confirm the interaction even in vivo, co-immunoprecipitation was performed in the cell to confirm the interaction between the 4 types of proteins and Rv3364c, and as a result, finally, SNX9 and YEATS4 were finally selected (Experiment Result 1).

SNX9 plays an important role in endocytosis by binding to a lipid bilayer and functions as an important signaling factor in bacterial infection and inflammatory responses. In order to search for a motif of Rv3364c involved in SNX9 binding, the present inventors prepared total 13 types of Tat-Rv3364c peptides by fragmenting Rv3364c into a peptide having 10 amino acids to fuse a cell-penetrating domain, that is, a transduction domain of an HIV-1 Tat protein for cell penetration to an N-terminus of each peptide and observed the interaction with SNX9. As a result, it was confirmed that the Tat-Rv3364c (₁₁DWLVSKFARE₂₀) peptide blocked the binding of SNX9 and Rv3364c and 11-20 a.a of Rv3364c was a region involved in binding to SNX9, and even among 11-20 a.a, particularly, a region of 12-17 a.a was a region required for interaction with SNX9 (Experimental Result 2).

Next, the present inventors tried to confirm an effect of the Tat-Rv3364c (12WLVSKF17) peptide on an immune response in a host cell through interaction with SNX9. Unlike that rRv3364c induced TLR4-mediated inflammatory responses in macrophages, it was confirmed that a Tat-Rv3364c (₁₂WLVSKF₁₇) peptide inhibited TLR4-mediated inflammatory responses, and it was confirmed that the Tat-WLVSKF functioned as a negative modulator that blocked PI3K, MAPK, and NF-κB signal pathways of TLR4 ligand induction (Experimental Result 3).

Furthermore, the present inventors tried to confirm the action of Tat-WLVSKF in an endosome from the function of SNX9 involved in endocytosis. Under LPS stimulation, SNX9 interacts with p47phox in an early endosome to induce ROS production and inflammatory responses. Meanwhile, it was confirmed that Tat-WLVSKF inhibited ROS production and inflammatory responses by blocking the binding of SNX9 and p47phox in the early endosome (Experiment Result 4).

The present inventors confirm that the Rv3364c protein of Mycobacterium tuberculosis may block a TLR4-mediated inflammatory response after SNX9 using a motif interacting with SNX9, and is intended to provide a peptide consisting of a region of 12-17 a.a. of Rv3364c interacting with the SNX9 or including the same for the treatment of sepsis.

In the present disclosure, if the Rv3364c-derived peptide provided for the treatment of sepsis contains a 12-17 amino acid region of the Rv3364c protein and does not affect the functional properties of W₁₂ and F₁₇ amino acids with hydrophobic non-polar side chains, the Rv3364c-derived peptide may contain one or two or more additional amino acids at an N-terminus and/or C-terminus thereof. The peptide containing the 12-17 amino acid region of the Rv3364c protein may be obtained by fragmenting the Rv3364 protein or may be prepared by chemical synthesis methods, genetic engineering methods, etc. known in the art.

In the present specification, an amino acid sequence is listed in the order of N-terminus to C-terminus, and the amino acids constituting the Rv3364c-derived peptide interacting with SNX9 of the present disclosure may each independently be in an L-form or a D-form, and each amino acid may be an amino acid analog, a radiolabeled amino acid, or a fluorescently tagged amino acid.

In addition, the Rv3364c-derived peptide of the present disclosure may induce modification of an amino (N-) terminus or a carboxy (C-) terminus in order to select a partial site of the amino acid sequence and increase its activity. Through this modification, the peptide of the present disclosure may have an increased half-life upon in vivo administration.

Meanwhile, the peptide for treating sepsis of the present disclosure may further include a cell-penetrating peptide to the N-terminus of the above-described Rv3364c-derived peptide in order to increase cell permeability.

The cell-penetrating peptides (CPP) may be selected from the group consisting of HIV-TAT (SEQ ID NO: 15), TAT (SEQ ID NO: 16), dNP2 (SEQ ID NO: 17), VP22 (SEQ ID NO: 18), MPG (SEQ ID NO: 19), PEP-1 (SEQ ID NO: 20), EB1 (SEQ ID NO: 21), transportan (SEQ ID NO: 22), p-Antp (SEQ ID NO: 23), hCT(18-32) (SEQ ID NO: 24), KLA (SEQ ID NO: 25), and oligoarginine (SEQ ID NO: 26), and for example, an HIV-TAT sequence (GRKKRRQRRRPG; SEQ ID NO: 15) may be used. The HIV-TAT sequence is a sequence derived from human immunodeficiency virus-1, and has amino acids having a positive charge at a high frequency.

In addition, the amino terminus of the peptide for treating sepsis of the present disclosure may be bound with a protecting group, such as an acetyl group, a fluorenyl methoxycarbonyl group, a formyl group, a palmitoyl group, a myristyl group, a stearyl group, and polyethylene glycol (PEG), and the carboxy terminus of the peptide may be modified with a hydroxyl group (—OH), an amino group (—NH₂), an azide (—NHNH₂), or the like. In addition, the terminus of the peptide of the present disclosure or an R-group of the amino acid may be bound with fatty acids, oligosaccharides chains, all nanoparticles (gold particles, liposomes, heparin, hydrogel, etc.), amino acids, carrier proteins, and the like. The modification of the amino acids described above serves to improve the potency and stability of the peptide of the present disclosure. As used herein, the term “stability” refers not only to in vivo stability, but also to storage stability (including storage stability at room temperature, refrigeration, and frozen storage).

The present disclosure provides a peptide for treating sepsis including or consisting of an amino acid sequence of an Rv3364c region (12-17a.a) that interacts with SNX9.

The peptide for treating sepsis of the present disclosure may be administered parenterally during clinical administration and may be used in the form of general pharmaceutical preparations. Parenteral administration may refer to administration through routes other than oral, such as rectal, intravenous, peritoneal, intramuscular, arterial, transdermal, nasal, inhalation, ocular, and subcutaneous routes. When the peptide for treating sepsis of the present disclosure is used as a pharmaceutical, the peptide may additionally contain one or more active ingredients exhibiting the same or similar function.

That is, the peptide for treating sepsis of the present disclosure may be administered in various parenteral formulations, and for formulations, the peptide is formulated using commonly used diluents or excipients, such as a filler, an extender, a binder, a wetting agent, a disintegrant, and a surfactant. Formulations for parenteral administration include a sterile aqueous solution, a non-aqueous solution, a suspension, an emulsion, a lyophilizing agent, and a suppository. As the non-aqueous solution and the suspension, propylene glycol, polyethylene glycol, vegetable oil such as olive oil, injectable ester such as ethyl oleate, and the like may be used. As a base compound of the suppository, witepsol, macrogol, tween 61, cacao butter, laurinum, glycerogelatin, and the like may be used.

In addition, the peptide for treating sepsis of the present disclosure may be used in combination with many pharmaceutically acceptable carriers, such as physiological saline or an organic solvent, and in order to increase the stability or absorbability, carbonates such as glucose, sucrose, or dextran, antioxidants such as ascorbic acid or glutathione, chelating agents, low molecular weight proteins, or other stabilizers may be used as drugs.

The peptide for treating sepsis of the present disclosure may be administered to a patient in a single dose in a bolus form or by infusion and the like for a relatively short period of time, and may be administered in a multiple dose for a long period of time. The administration form and period are determined in consideration of various factors such as the age and health condition of the patient as well as the route of administration and the number of treatments of the drug. Thus, considering this point, those skilled in the art may determine an appropriate effective dosage of the peptide for treating sepsis of the present disclosure.

Since the peptide for treating sepsis of the present disclosure is excellent in the treatment effect of sepsis, the peptide for treating sepsis may be used in the preparation of a pharmaceutical composition for treating sepsis.

Accordingly, the present disclosure provides a pharmaceutical composition for treating sepsis including the peptide for treating sepsis as an active ingredient.

In addition, the present disclosure may provide a method for preventing or treating sepsis or septic shock including administering the pharmaceutical composition to a subject, in which the subject is not limited as long as it is any mammal infected with a causative organism causing sepsis or suspected of being infected therewith, but may preferably be humans or livestock.

Since the pharmaceutical composition for treating sepsis contains the peptide for treating sepsis described above as an active ingredient, the duplicated descriptions will be omitted.

As used herein, the term “treatment” refers to any action in which the symptoms of sepsis are improved or beneficially changed by administration of the peptide according to the present disclosure or the pharmaceutical composition including the same.

As used herein, the term “containing as the active ingredient” refers to a sufficient amount to treat the disease at a reasonable benefit/risk ratio applicable to medical treatment, and an effective dose level may be determined according to factors including the type and severity of disease of a patient, the activity of a drug, the sensitivity to a drug, a time of administration, a route of administration, and an excretion rate, duration of treatment, and simultaneously used drugs, and other factors well-known in the medical field. The peptide according to the present disclosure or the pharmaceutical composition including the same may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side-effects by considering all the factors, which may be easily determined by those skilled in the art. The dosage and the number of administrations of the pharmaceutical composition of the present disclosure are determined according to a type of drug as an active ingredient in addition to many related factors, such as a route of administration, the age, sex, and weight of a patient, and the severity of diseases.

Accordingly, the pharmaceutical composition according to the present disclosure may include various pharmaceutically acceptable carriers as long as the peptide according to the present disclosure is contained as an active ingredient.

As the pharmaceutically acceptable carrier, in oral administration, a binder, a lubricant, a disintegrant, an excipient, a solubilizer, a dispersant, a stabilizer, a suspending agent, a pigment, a flavoring, and the like may be used. In the case of injections, a buffering agent, a preservative, a painless agent, a solubilizer, an isotonic agent, a stabilizer, and the like may be used in combination. In the case of topical administration, a base, an excipient, a lubricant, a preservative, and the like may be used. The formulations of the pharmaceutical composition of the present disclosure may be prepared variously in combination with the pharmaceutically acceptable carriers described above. For example, for oral administration, the pharmaceutical composition of the present disclosure may be prepared in the form of tablets, troches, capsules, elixirs, suspensions, syrups, wafers, and the like, and for injectable administration, the pharmaceutical composition of the present disclosure may be prepared in the form of a single dose ampoule or a multiple dose form. In addition, the pharmaceutical composition of the present disclosure may be also formulated into other solutions, suspensions, tablets, pills, capsules, sustained release agents, and the like.

Meanwhile, examples of the carrier, the excipient, and the diluent suitable for the formulations may include lactose, dextrose, sucrose, sorbitol, mannitol, xylitol, erythritol, maltitol, starch, acacia, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oils, or the like. In addition, the pharmaceutical composition may further include fillers, anti-coagulating agents, lubricants, wetting agents, flavorings, antiseptics, and the like.

Meanwhile, the present disclosure provides a food composition for preventing or improving sepsis including the peptide for treating sepsis as an active ingredient.

Since the food composition uses the peptide for treating sepsis, the duplicated contents between the two will be omitted to avoid excessive description of the specification.

In the present disclosure, the food composition may be provided in the form of powders, granules, tablets, capsules, syrups, beverages, or pills, and may be used together with other foods or food additives in addition to the peptide for treating sepsis as the active ingredient, and may be appropriately used according to a conventional method. The mixed amount of the active ingredients may be suitably determined according to the purpose of use thereof, for example, the prevention, health, or therapeutic treatment.

The effective dose of the active ingredient contained in the food composition may be used according to an effective dose of the pharmaceutical composition, but may be the range or less in the case of long-term intake for health and hygiene purposes or for health control, and it is certain that the active ingredient may be used even in an amount above the range because there is no problem in terms of safety.

In addition, the food composition may include ingredients commonly added during food preparation, and includes, for example, proteins, carbohydrates, fats, nutrients, seasonings, and flavoring agents. Examples of the carbohydrates may include monosaccharides, for example, glucose, fructose, and the like; disaccharides, for example, maltose, sucrose, oligosaccharide, and the like; and polysaccharides, for example, general sugars such as dextrin and cyclodextrin and sugar alcohols such as xylitol, sorbitol, and erythritol. As the flavoring agents, natural flavoring agents and synthetic flavoring agents may be used. For example, when the food composition of the present disclosure is prepared in the form of drinks, citric acid, liquid fructose, sugar, glucose, acetic acid, malic acid, fruit juice, etc. may be additionally included in addition to the active ingredient of the present disclosure.

In the present disclosure, an amino acid sequence is abbreviated as follows according to the IUPAC-IUB nomenclature:

Arginine (Arg, R), lysine (Lys, K), histidine (His, H), serine (Ser, S), threonine (Thr, T), glutamine (Gln, Q), asparagine (Asp, N), methionine (Met, M), leucine (Leu, L), isoleucine (Ile, I), valine (Val, V), phenylalanine (Phe, F), tryptophan (Trp, W), tyrosine (Tyr, Y), alanine (Ala, A), glycine (Gly, G), proline (Pro, P), cysteine (Cys, C), aspartic acid (Asp, D), glutamic acid (Glu, E), norleucine (Nle).

Hereinafter, example embodiments will be described in detail with reference to the accompanying drawings. However, since various modifications may be made to the example embodiments, the scope of the present disclosure is not limited or restricted by these example embodiments. It should be understood that all modifications, equivalents, and substitutes for the example embodiments are included in the scope of the present disclosure.

The terms used in the example embodiments are used for the purpose of description only, and should not be construed to be limited. The singular expression includes the plural expression unless the context clearly dictates otherwise. In the present application, it should be understood that term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which example embodiments pertain. Terms which are defined in a generally used dictionary should be interpreted to have the same meaning as the meaning in the context of the related art, and are not interpreted as ideal or excessively formal meanings unless otherwise defined in the present application.

In addition, in the description with reference to the accompanying drawings, like components designate like reference numerals regardless of reference numerals and a duplicated description thereof will be omitted. In describing the example exemplary embodiments, a detailed description of related known technologies will be omitted if it is determined that they unnecessarily make the gist of the example exemplary embodiments unclear.

[Experimental Methods and Materials]

1. Mouse and Cell Culture

Wild-type C57BL/6 mice were purchased from Samtako Bio Korea (Gyeonggi-do, Korea). Primary bone marrow-derived macrophages (BMDMs) were isolated from C57BL/6 mice and cultured in a DMEM added with M-CSF (R&D Systems, 416-ML) for 3 to 5 days. BMDM cells (TLR2^{-/-}, TLR4^{-/-}, MyD88^{-/-}, TRIF^{-/-}, IRAK1^{-/-}, TRAF6^{-/-}, and TBK1^{-/-}) of C57BL/6 mice were provided from Dr. Cheol-Ho Lee (Laboratory Animal Center, Korea Research Institute of Bioscience and Biotechnology; Daejeon, Korea) and SNX9^{-/-} C57BL/6 rats were provided from Dr. Zhigang Xu (Shandong Provincial Key Laboratory of Animal Cells and Developmental Biology, Shandong University, Jinan, P.R. China) to perform an experiment.

In a DMEM containing 10% FBS (Gibco), sodium pyruvate, nonessential amino acids, penicillin G (100 IU/ml), and streptomycin (100 IU/ml), HEK293T (ATCC-11268; American Type Culture Collection) and RAW264.7 (ATCC TIB-71) were cultured. Human monocyte THP-1 (ATCC TIB-202) cells were cultured in RPMI 1640/glutamax supplemented with 10% FBS and treated with 20 nM PMA (Sigma-Aldrich) for 24 hours to induce differentiation into macrophages and then washed 3 times with PBS. Transient transfection was performed using calcium phosphate (Clontech) in 293T according to manufacturer's instructions.

2. Preparation of Recombinant Protein

In order to prepare a recombinant Rv3364c (rRv3364c) protein, an MTB H37Rv strain Rv3364c (GenBank accession no. NP_217881) base sequence was cloned into a pRSFDuet-1 vector (Novagen) using an N-terminal 6xHis tag according to a manufacturer's recommended protocol, and expression-induced, harvested, and purified in an E. coli BL21(DE3)pLysS strain. The purified rRv3364c was dialyzed against a permeable cellulose membrane, and lipopolysaccharide (LPS) contamination was tested by Limulus amebocyte lysate assay (BioWhittaker). The following experiments contained rRv3364c and a mutant protein at concentrations of 20 pg/ml or less. The purified rRv3364c (13 kDa) was verified and used through SDS polyacrylamide gel electrophoresis and immunoblotting, and significant rRv3364c-induced cytotoxicity was not observed in macrophages.

3. Reagents and Antibodies

LPS (Escherichia coli 0111:B4) was purchased from Invivogen. Phospho-(Ser473)-AKT, phospho-(Thr202/Tyr204)-p42/44, phospho-(Thr180/Tyr 182)-p38, phospho-(Thr183/Tyr185)-SAPK/JNK, and phospho-(Ser32/36)-IκB-α-specific antibodies were purchased from Cell Signaling Technology (Danvers, Mass., USA). SNX9 (ab181856), YEATS4, (ab50963), ZBTB46 (ab277100), and EGFLAM (ab101398)-specific antibodies were purchased from Abcam plc. IκB-α (C-21), p22phox (FL-195), gp91phox (H-60), p47phox (H-195), p67phox (H-300), Lamin B1 (B-10), Tubulin (5F131), CD68 (KP1), F4/80 (BM8), CD3 (PC3/188A), CD19 (SJ25-C1), His (His17), Flag (D-8), V5(H-9), Myc (9E10), and Actin (I-19)-specific antibodies were purchased from Santa Cruz Biotechnology.

4. Design of Plasmids

SNX9, YEATS4, ZBTB46, and EGFLAM plasmids were purchased from Addgene. Plasmids encoding full-length p47phox and mutants thereof were prepared in the same manner as in previous studies (Biomaterials 2016, 89, 1-13). Plasmids cloning different regions 1-595, 1-250, 250-361, and 392-595 of SNX9 were prepared by amplifying each region from full-length SNX9 cDNA by PCR to be subcloned between BamHI and NotI regions of a pEF-IRES-Puro expression vector. Base sequences of all plasmids were confirmed using an ABI PRISM 377 automatic DNA sequencer.

5. Peptides

A Tat-binding Rv3364c peptide was synthesized by Peptron (Korea) and purified in the form of an acetate salt in order to avoid abnormal reactions in cells. The amino acid sequence of each peptide is shown in Table 1. Endotoxin content was measured using Limulus amebocyte lysate assay (BioWhtaker), and the peptides used in the following experiments were contained at a concentration of 3 to 5 pg/ml.

TABLE 1

| Rv3364c peptide | Amino acid sequence | SEQ ID NO: |
|---|---|---|
| Rv3364c(1-10 aa) | MKARLPDSPL | 1 |
| Rv3364c(11-20 aa) | DWLVSKFARE | 2 |
| Rv3364c(21-30 aa) | VPGVAHALLV | 3 |
| Rv3364c(31-40 aa) | SVDGLPVAAS | 4 |
| Rv3364c(41-50 aa) | EHLPRERADQ | 5 |
| Rv3364c(51-60 aa) | LAAVTSGLAS | 6 |
| Rv3364c(61-70 aa) | LAGGAAQLFD | 7 |
| Rv3364c(71-80 aa) | GGQVLQSVVE | 8 |
| Rv3364c(81-90 aa) | MQNGYLLLMQ | 9 |
| Rv3364c(91-100 aa) | VGDGSALAAL | 10 |
| Rv3364c(101-110 aa) | AATGCDIGQI | 11 |
| Rv3364c(111-120 aa) | GYEMAILVER | 12 |
| Rv3364c(121-130 aa) | VGGVVQSCRR | 13 |

6. Identification of rRv3364c Binding Proteins Using HuProt™ Microarray

In order to identify a protein binding to rRv3364c, human protein microarrays (CDI Labs, USA) containing 20,000 or more full-length recombinant human proteins were used. Specifically, the protein microarrays were treated with a blocking buffer (2% BSA in PBS, 0.1% in 20) for 2 hours. 3 μg of biotinylated was treated on the microarrays at 4° C. for 8 hours. Thereafter, each array was treated with 1 μg of streptavidin fluorescence Alexa Fluor 532 nm at 4° C. for 1 hour. Microarray results were detected using a GenePix 4100A microarray laser scanner (Molecular Devices, USA).

7. Enzyme-Linked ImmunoSorbent Assay (ELISA)

Cytokines (TNF-α, IL-6, IL-1β, IL-18, IL-12p40, and IL-10) were measured in a cell culture supernatant and a rat serum using a BD OptEIA ELISA set (BD Pharmingen) according to a manufacturer's recommended protocol. All experiments

8. Production of CLP-Induced Sepsis Animal and Bacterial Counting

Cecal ligation and puncture (CLP) was performed on 6-week-old C57BL/6 female mice (Samtako Bio, Gyeonggi-do, Korea). Specifically, the mice were anesthetized with pentothal sodium (50 mg/kg, i.p.) and a small abdominal midline incision was made to expose the cecum. Thereafter, the cecum was connected under the ileocecal valve, the surface was punctured twice with a 22-gauge needle, and the abdomen was sutured. The survival rate of mice was checked daily for 10 days. After 12 hours and 24 hours after the CLP was performed, the mice were intraperitoneally injected with PBS, an analgesic (1.5 mg/kg nalbuphine; Sigma-Aldrich), and an antibiotic cocktail. The antibiotic cocktail contained ceftriaxone (25 mg/kg; Sigma-Aldrich) and metronidazole (12.5 mg/kg; Sigma-Aldrich) in 100 μl PBS. As a control group (Sham), mice subjected to only the cecal exposure surgery without ligation and puncture were used.

The number of bacteria was confirmed as follows. After performing CLP, blood was collected from the cardiac cavity or peritoneal fluid of mice at a fixed time, and the collected blood was continuously diluted. Thereafter, 5 μl of each dilution was spread on a blood agar plate and cultured at 37° C. for 24 hours. The number of bacteria was calculated by counting colony-forming units (CFU) per total peritoneal washing fluid or blood.

All animals were bred in a pathogen-free environment, and all experimental procedures were performed under review and approval of the Animal Protection and Utilization Committee (protocol 2020-0060) of Hanyang University. After the CLP was performed, administration of analgesics, antibiotics, and fluids was performed according to the international guidelines defined in “Minimum Quality Threshold in Pre-Clinical Sepsis Studies”.

9. Histological Analysis

For tissue analysis, the spleen, liver, and lungs of mice were fixed in 10% formalin and placed in paraffin. Paraffin sections (4 μm) were cut and stained with hematoxylin and eosin (H&E). Histopathological scores (0 to 4) were scored independently for each organ section by a pathologist based on the number and distribution of inflammatory cells in the tissue and the severity of inflammation without prior knowledge of a treatment group.

10. In Vivo Images

FL-DATPT was prepared by adding a Cy5.5 dye bound with streptavidin to DATPT. FL-DATPT was administered intraperitoneally (i.p) to CLP mice. After administration of FL-DATPT, mice were sacrificed at different time points and major organs were excised and then imaged using an IVIS Spectrum-CT in vivo imaging system (PerkinElmer, Inc.) to observe the vivo distribution in the tissue.

11. Statistical Analysis

All data were analyzed using a Student's t-test with Bonferroni adjustment or ANOVA for multiple comparisons and disclosed as mean ±SD. Statistical analysis was performed with an SPSS (Version 12.0) statistical software program (SPSS, Chicago, Ill., USA). Differences were considered significantly at p<0.05. Survival rates were analyzed by using GraphPad Prism (version 5.0, La Jolla, Calif., USA), and using a log-rank (Mantele-Cox) test for comparison to graph data using a product limit method of Kaplan and Meier.

[Experiment Results]

1. Confirmation of Direct Interaction of Rv3364c with SNX9 and YEATS4

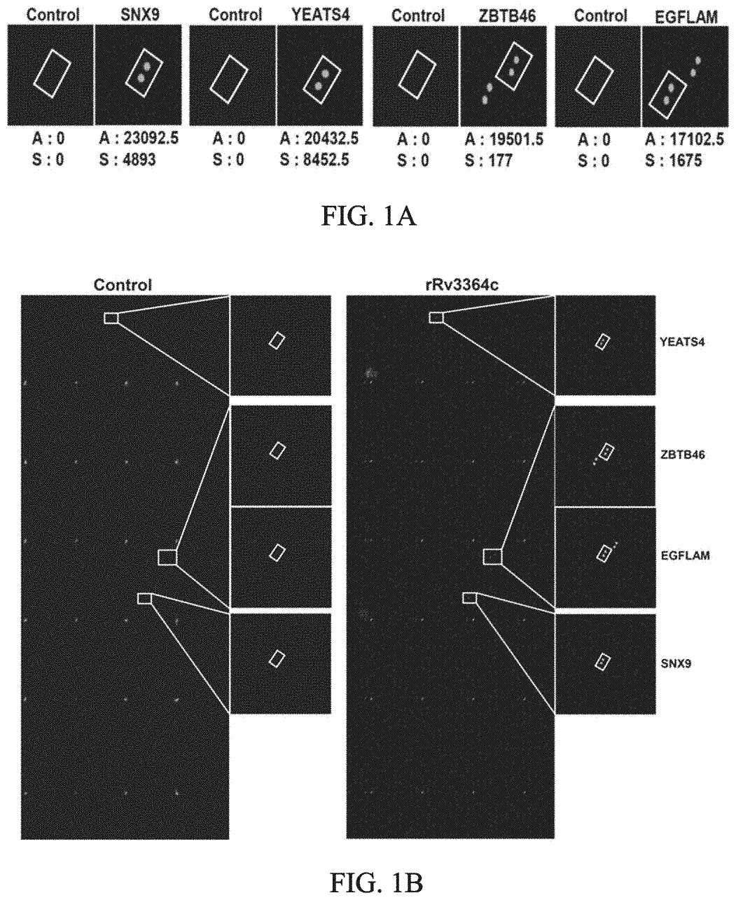

rRv3364c binds to cathepsin G, a membrane protein of macrophages, to inhibit its enzymatic activity and inhibit caspase-1-dependent apoptosis activation, which is a sub-step thereof. In order to identify factors interacting with rRv3364c in a host cell, the interaction with a specific protein was observed in a cell-free system. A customized protein binding assay was used to identify interacting proteins, and candidate proteins capable of potentially interacting with rRv3364c were selected. Among 118 candidate proteins predicted to bind to rRv3364c, SNX9 (sorting nexin 9), YEATS4 (YEATS domain-containing protein 4), ZBTB46 (zinc finger and BTB domain-containing 46), and EGFLAM (EGF-like Fibronectin type III and Laminin G domains) showed high affinity with rRv3364c (FIGS. 1A and 1B). In FIG. 1A, an affinity score (A score) is a normalized signal intensity of two overlapping points, and a specificity score (B score) is a difference between the affinity score of the protein and the protein ranked next thereto.

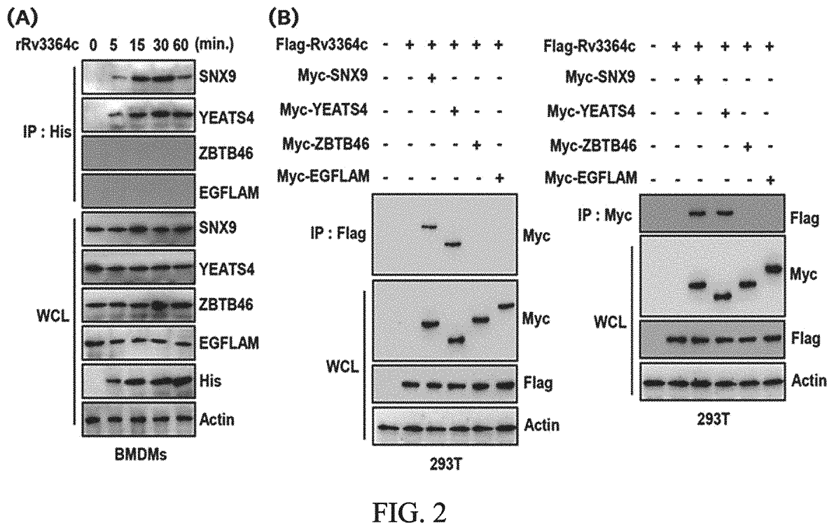

Temporarily (15 to 30 min), co-immunoprecipitation in macrophages and HEK293T cells was performed to analyze proteins interacting with rRv3364c in vivo, and as a result, it was confirmed that rRv3364c strongly interacted with endogenous and extrinsic SNX9 and YEATS4, unlike ZBTB46 and EGFLAM (FIG. 2).

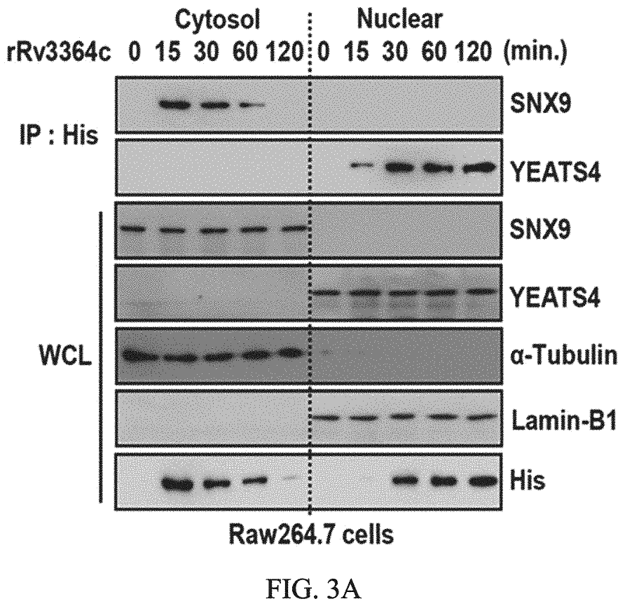

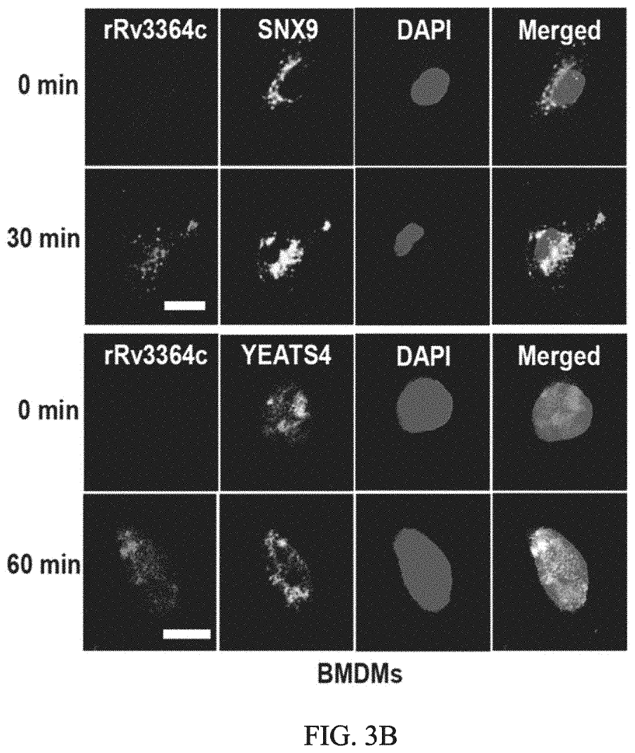

SNX9 binds to a lipid bilayer to regulate endocytosis and migration of YEATS4 to the nucleus, and cell fractionation and immunostaining analysis were performed to confirm the intracellular position of rRv3364c in macrophages. As a result, it was confirmed that rRv3364c was initially bound to SNC9 in the cytoplasm and then migrated to the nucleus to bind to YEATS4 (FIGS. 3A and 3B). From the above results, it can be seen that rRv3364c directly interacts with SNX9 in the cytoplasm and YEATS4 in the nucleus in a time-dependent manner.

2. Confirmation of Regions of Rv3364c Required for Interaction with SNX9 or YEATS4

Next, a peptide sequence of Rv3364c involved in the interaction with SNX9 or YEATS4 was confirmed. An Rv3364c-derived peptide was designed by cutting Rv3364c with a length of 10 a.a. (Table 1), and in order to prevent intracellular migration and protein degradation, a retro-inverso peptide (Tat-Rv3364c peptide) was formed by fusing a transduction domain of an HIV-1 Tat protein. Then, the interaction of the peptide fragment with SNX9 and YEATS4 in HEK293T cells was confirmed.

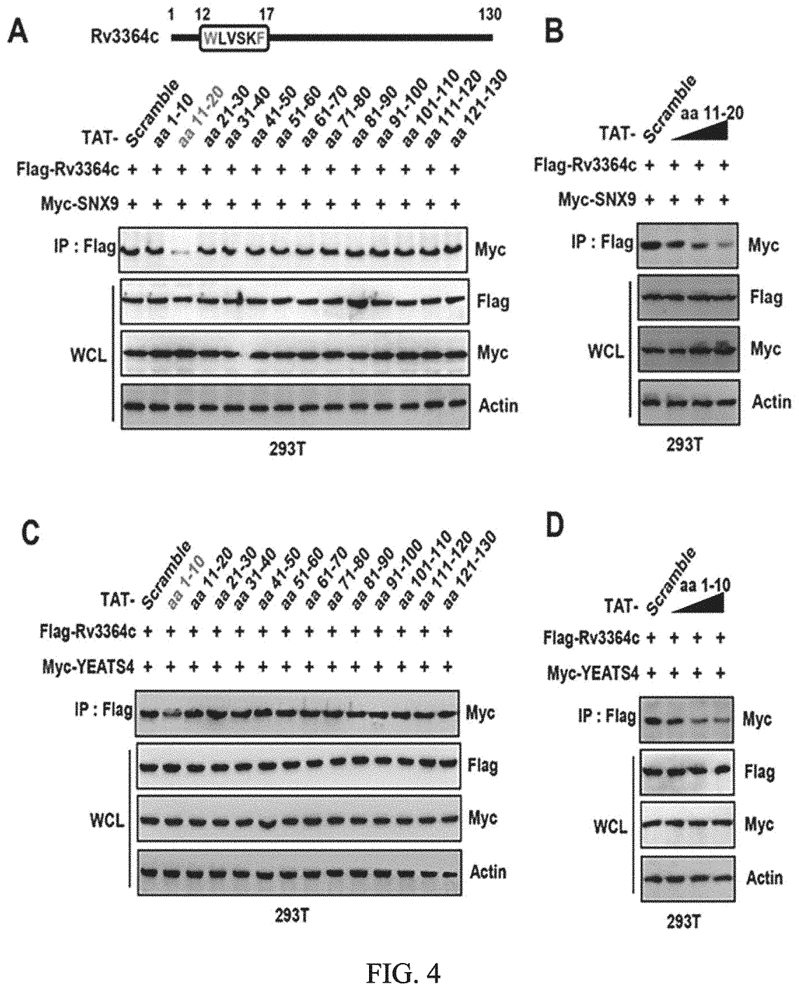

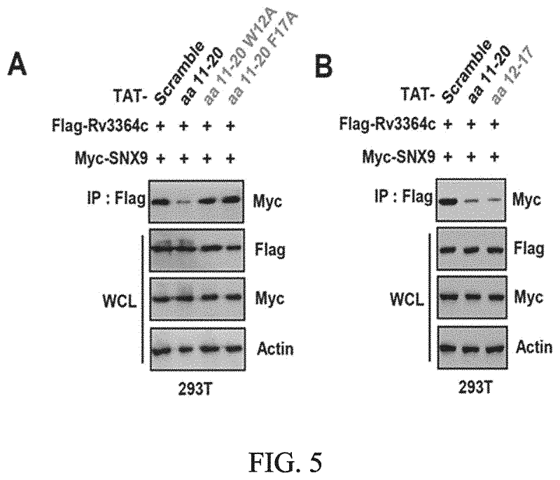

As a result, a Tat-Rv3364c (₁₁DWLVSKFARE₂₀) peptide may block the interaction between SNX9 and Rv3364c, and a Tat-Rv3364c (₁MKARLPDSPL₁₀) may block the interaction between YEATS4 and Rv3364c, and the Tat peptide had no effect on this interaction (FIG. 4). In addition, it could be seen that a minimum peptide essential for interaction with SNX9 was ₁₂WLVSKF₁₇ (SEQ ID NO: 14), and amino acids W₁₂ and F₁₇ having hydrophobic non-polar side chains were essential for interaction with SNX9 (FIG. 5).

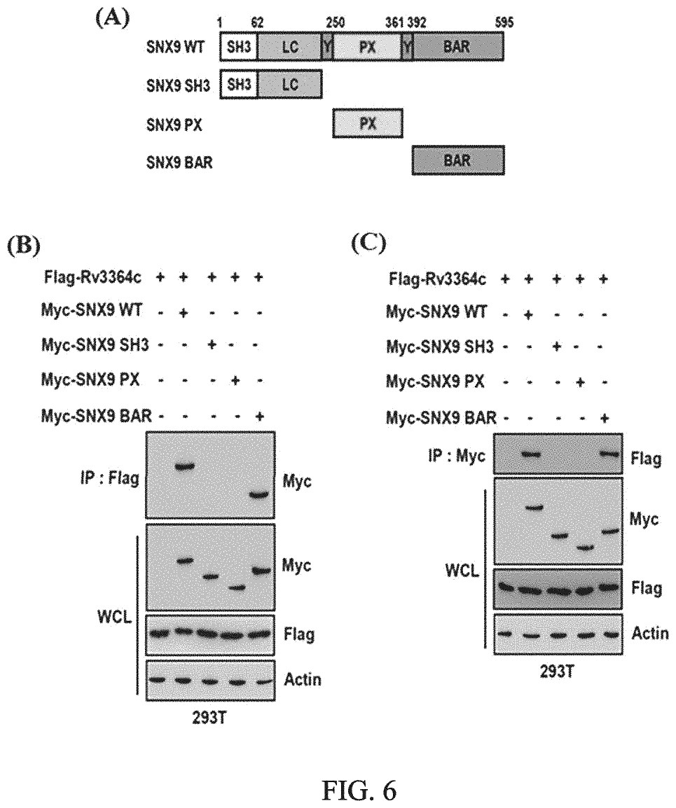

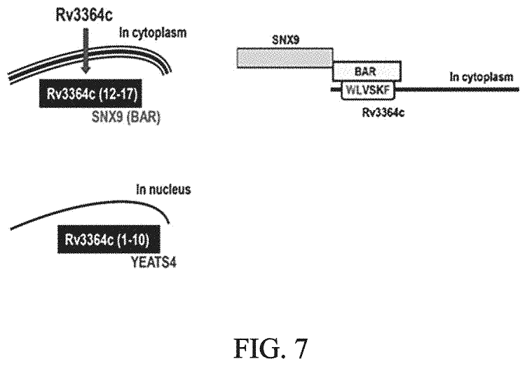

Subsequently, as a result of preparing various cleavage mutants of SNX9 and confirming a domain of SNX9 interacting with Rv3364c, it was found that Rv3364c interacted with SNX9 through a Bin-Amphiphysin-Rvs (BAR) domain (FIG. 6). From the results, it can be seen that in Rv3364c, a 12-17 a.a. peptide (SEQ ID NO: 14) in the cytoplasm specifically targets a BAR domain through an N-terminus of SNX9, and a 1-10 a.a. peptide in the nucleus interacts with YEATS4 (FIG. 7).

3. Confirmation of Negative Regulatory Function of TLR4 Signaling Pathway Through Interaction of WLVSKF Peptide with SNX9

The production levels of rRv3364c-induced cytokines were measured in macrophages using ELISA.

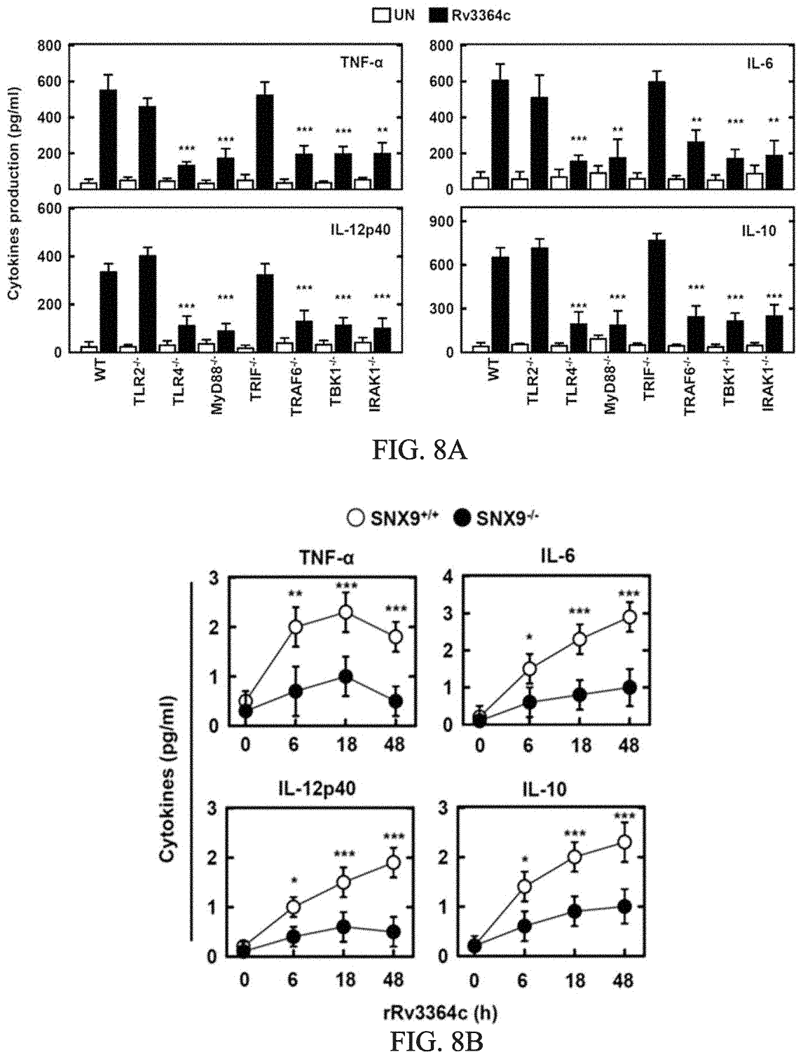

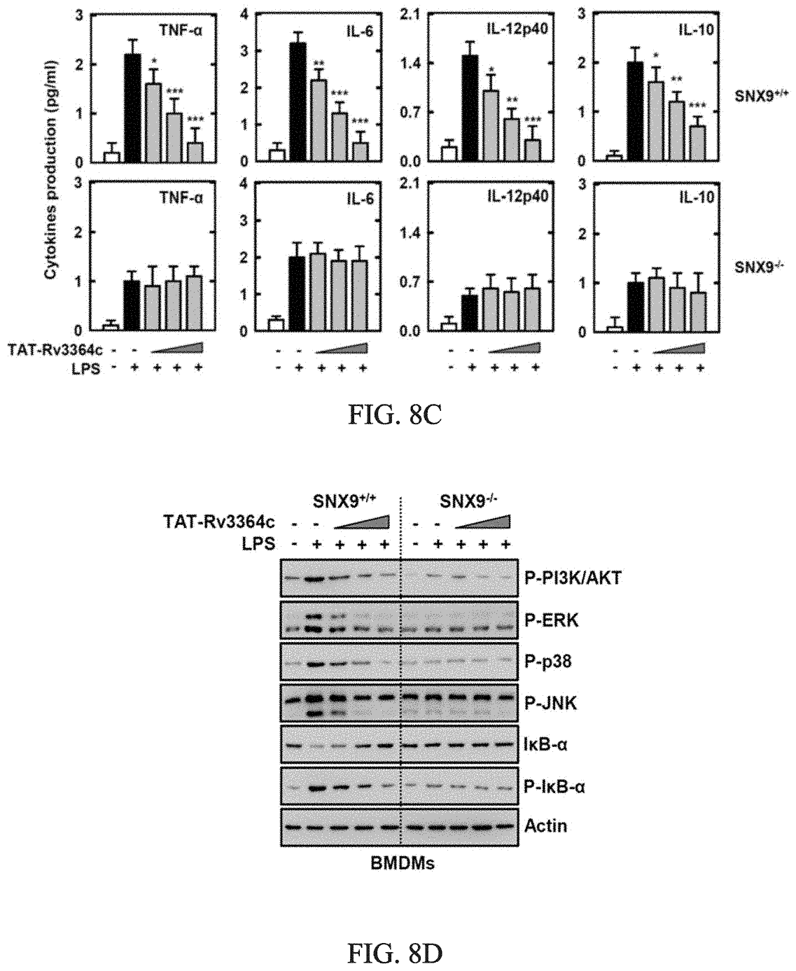

It was confirmed that rRv3364c promoted the production of pro-inflammatory cytokines (TNF-α, IL-6, and IL-12p40) and anti-inflammatory cytokines (IL-10) through a TLR4/MyD88/TRAF6/TBK1-IRAK1 pathway (FIG. 8A). In addition, the production of rRv3364c-induced inflammatory cytokines was significantly reduced in SNX9^{-/-} macrophages compared to SNX9^{+/+} macrophages (FIG. 8B). From the above, it can be seen that a TLR4-SNX9-dependent inflammatory response is induced by rRv3364c in macrophages.

Next, in order to confirm the role of the rRv3364c peptide (₁₂WLVSKF₁₇) interacting with SNX9 in the TLR-mediated inflammatory signaling pathway, it was confirmed whether the Tat-Rv3364c peptide, Tat-WLVSKF, inhibited the production of LPS/TLR-induced inflammatory cytokines. As a result, it was confirmed that, unlike in SNX9^{-/-} macrophages, the production of TLR4-mediated inflammatory cytokines was reduced by Tat-WLVSKF in SNX9^{+/+} macrophages (FIG. 8C).

From the above, it is assumed that rRv3364c regulates the TLR4 signaling pathway in cells, and to verify this, the effect of Tat-WLVSKF on the activation of PI3K, MAPK, and NF-κB involved in LPS-induced inflammatory signaling in macrophages was confirmed. As a result, unlike in SNX9^{-/-} macrophages, it was confirmed that Tat-WLVSKF pretreatment inhibited LPS-induced phosphorylation of AKT, MAPK, and IκBα and degradation of IκBα in SNX9^{+/+} macrophages (FIG. 8D). These results suggest that Tat-WLVSKF serves as a negative regulator to inhibit TLR4 ligand-induced activation of PI3K, MAPK, and NF-κB signaling pathways, thereby inhibiting the production of inflammatory cytokines in macrophages.

4. Confirmation of BAR Domain Inhibitory Activity of SNX9 Interacting with p47phox in Endosome of WLVSKF Peptide

NADPH oxidase (NOX)-induced ROS production is essential for TLR4-mediated immune responses. In particular, p47phox, a cytoplasmic NOX subunit, moves through a PX (Phox homology) domain in the plasma membrane upon infection to bind to an NOX subunit. The PX domain of SNX9 reacts with various phosphoinositides and plays an important role in endocytosis.

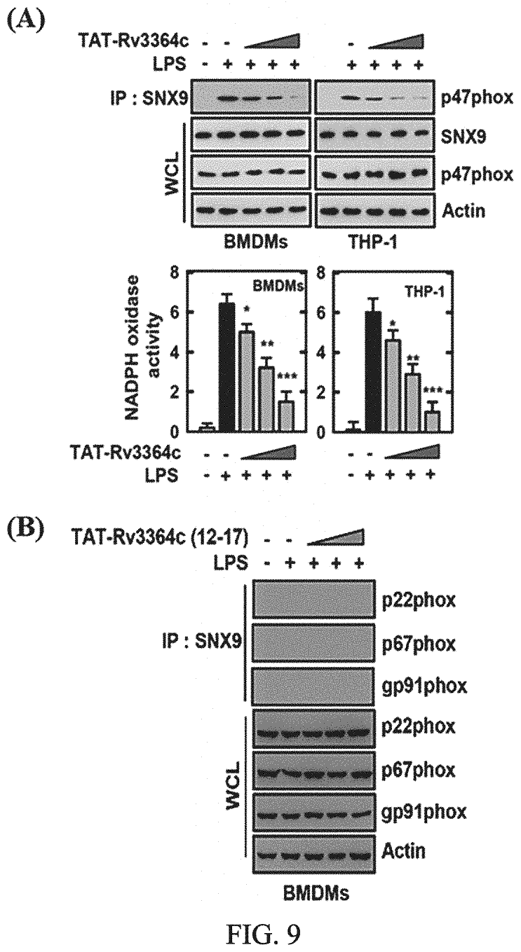

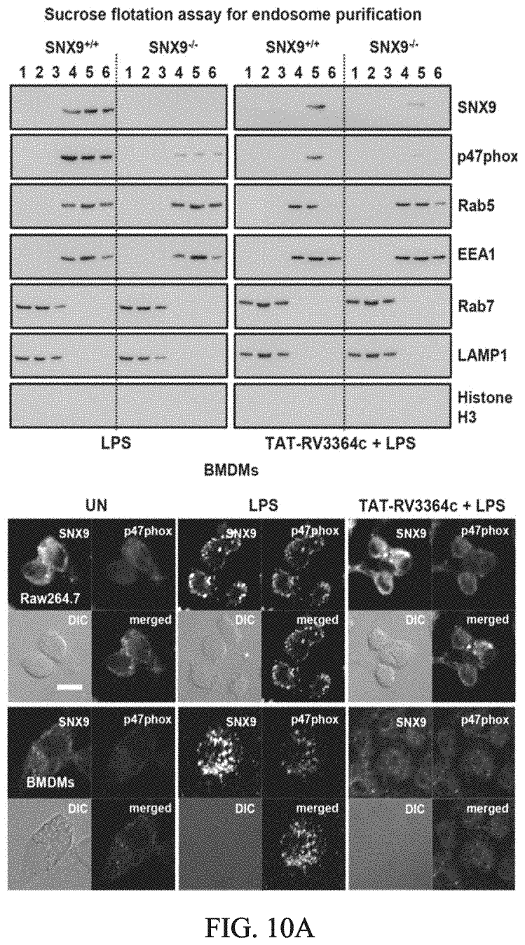

Hereinafter, it was intended to determine whether SNX9 and p47phox interact with each other under LPS stimulation and whether Tat-WLVSKF affects endosomal ROS production. As a result, it was confirmed that SNX9 was specifically bound to p47phox under LPS induction, and the interaction between SNX9 and p47phox was inhibited by Tat-WLVSKF. In addition, in mouse and human macrophages, NOX activity and ROS production were significantly decreased in a Tat-WLVSKF dose-dependent manner (FIG. 9).

In addition, the movement of SNX9 in endosomes was tracked and signaling and molecular dynamics in endosomes were observed by centrifuging and purifying sections of endosomes by sucrose flotation gradient assay and performing immunofluorescence microscopy. As a result, LPS-induced binding of SNX9 and p47phox was confirmed in an early endosome and not observed in a late endosome, and it was confirmed that LPS-induced binding of SNX9 and p47phox in the early endosome was inhibited by Tat-WLVSKF (FIGS. 10A to 10C).

Subsequently, it was confirmed that a PX domain of p47phox was essential for binding to the BAR domain of SNX9 by mapping using various cleaved forms of p47phox and SNX9 in HEK293T cells (FIGS. 11A-11B). In addition, as a result of competition analysis using the Rv3364c structure, it was confirmed that the interaction between SNX9 and p47phox decreased as the amount of Rv3364c increased in HEK293T cells (FIG. 12). From the above results, it can be seen that SNX9 is an essential positive regulator of the ROS-mediated inflammatory response through the binding to the PX domain of p47phox in the early endosome, and Tat-WLVSKF blocks the binding of SNX9 and p47phox in the early endosome to inhibit the ROS-mediated inflammatory response. Ultimately, it can be seen that the Tat-WLVSKF peptide derived from Rv3364c may function as a potent inflammatory regulator by specifically inhibiting an LPS/TLR4-mediated immune response in macrophages (FIG. 13).

5. Confirmation of Sepsis Treatment Effect of WLVSKF Peptide in CLP-Induced Sepsis Animal Model

Next, it was attempted to confirm the effect of the peptide derived from Rv3364c in CLP-induced sepsis mice. After 0, 1, and 2 hours, 20 mg/kg of Tat-Rv3364c was intraperitoneally injected into sepsis-induced mice treated with CLP. As a result, it was confirmed that the survival rate increased to 50% when Tat-Rv3364c was administered (FIG. 14).

As described above, although the example embodiments have been described by the restricted drawings, various modifications and variations can be applied on the basis of the example embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other example embodiments, and equivalents to the appended claims fall within the scope of the claims to be described below.

What is claimed is:

1. A peptide for treating sepsis comprising an amino acid sequence represented by SEQ ID NO: 14.

2. The peptide for treating sepsis of claim 1, wherein the peptide further includes 1 to 3 amino acids to an N-terminus and/or a C-terminus.

3. The peptide for treating sepsis of claim 2, wherein the peptide consists of an amino acid sequence represented by SEQ ID NO: 2.

4. The peptide for treating sepsis of claim 1, wherein a cell-penetrating peptide is linked to an N-terminus of the peptide for treating sepsis.

5. The peptide for treating sepsis of claim 4, wherein the cell-penetrating peptide is one type selected from the group consisting of HIV-TAT (SEQ ID NO: 15), TAT (SEQ ID NO: 16), dNP2 (SEQ ID NO: 17), VP22 (SEQ ID NO: 18), MPG (SEQ ID NO: 19), PEP-1 (SEQ ID NO: 20), EB1 (SEQ ID NO: 21), transportan (SEQ ID NO: 22), p-Antp (SEQ ID NO: 23, hCT(18-32) (SEQ ID NO: 24), KLA (SEQ ID NO: 25), and oligoarginine (SEQ ID NO: 26.

6. The peptide for treating sepsis of claim 1, wherein the peptide directly interacts with a BAR domain of SNX9 in macrophages.

7. The peptide for treating sepsis of claim 1, wherein the peptide inhibits a TLR4-mediated inflammatory response in macrophages.

8. The peptide for treating sepsis of claim 1, wherein the peptide interferes with binding of SNX9 and p47phox to reduce ROS production.

9. A pharmaceutical composition for preventing or treating sepsis comprising the peptide of claim 1 as an active ingredient.

10. A food composition for preventing or improving sepsis comprising the peptide of claim 1 as an active ingredient.

11. A method of preventing or treating sepsis comprising administering the peptide of claim 1 to a subject.


SEQUENCE LISTING (26 sequences)

SEQ ID NO: 1 - protein, 10 aa, synthetic construct
  REGION 1..10: note = Rv3364c(1-10 aa)
MKARLPDSPL

SEQ ID NO: 2 - protein, 10 aa, synthetic construct
  REGION 1..10: note = Rv3364c(11-20 aa)
DWLVSKFARE

SEQ ID NO: 3 - protein, 10 aa, synthetic construct
  REGION 1..10: note = Rv3364c(21-30 aa)
VPGVAHALLV

SEQ ID NO: 4 - protein, 10 aa, synthetic construct
  REGION 1..10: note = Rv3364c(31-40 aa)
SVDGLPVAAS

SEQ ID NO: 5 - protein, 10 aa, synthetic construct
  REGION 1..10: note = Rv3364c(41-50 aa)
EHLPRERADQ

SEQ ID NO: 6 - protein, 10 aa, synthetic construct
  REGION 1..10: note = Rv3364c(51-60 aa)
LAAVTSGLAS

SEQ ID NO: 7 - protein, 10 aa, synthetic construct
  REGION 1..10: note = Rv3364c(61-70 aa)
LAGGAAQLFD

SEQ ID NO: 8 - protein, 10 aa, synthetic construct
  REGION 1..10: note = Rv3364c(71-80 aa)
GGQVLQSVVE

SEQ ID NO: 9 - protein, 10 aa, synthetic construct
  REGION 1..10: note = Rv3364c(81-90 aa)
MQNGYLLLMQ

SEQ ID NO: 10 - protein, 10 aa, synthetic construct
  REGION 1..10: note = Rv3364c(101-110 aa)
VGDGSALAAL

SEQ ID NO: 11 - protein, 10 aa, synthetic construct
  REGION 1..10: note = Rv3364c(101-110 aa)
AATGCDIGQI

SEQ ID NO: 12 - protein, 10 aa, synthetic construct
  REGION 1..10: note = Rv3364c(111-120 aa)
GYEMAILVER

SEQ ID NO: 13 - protein, 10 aa, synthetic construct
  REGION 1..10: note = Rv3364c(121-130 aa)
VGGVVQSCRR

SEQ ID NO: 14 - protein, 6 aa, synthetic construct
  REGION 1..6: note = Rv3364c(12-17 aa)
WLVSKF

SEQ ID NO: 15 - protein, 12 aa, synthetic construct
  REGION 1..12: note = HIV-TAT
GRKKRRQRRRPQ

SEQ ID NO: 16 - protein, 11 aa, synthetic construct
  REGION 1..11: note = TAT
YGRKKRRQRRR

SEQ ID NO: 17 - protein, 24 aa, synthetic construct
  REGION 1..24: note = dNP2
KIKKVKKKGRKGSKIKKVKKKGRK

SEQ ID NO: 18 - protein, 34 aa, synthetic construct
  REGION 1..34: note = VP22
DAATATRGRSAASRPTERPRAPARSASRPRRPVE

SEQ ID NO: 19 - protein, 27 aa, synthetic construct
  REGION 1..27: note = MPG
GALFLGFLGAAGSTMGAWSQPKKKRKV

SEQ ID NO: 20 - protein, 21 aa, synthetic construct
  REGION 1..21: note = PEP-1
KETWWETWWTEWSQPKKKRKC

SEQ ID NO: 21 - protein, 23 aa, synthetic construct
  REGION 1..23: note = EB1
LIKLWSHLIHIWFQNRRLKWKKK

SEQ ID NO: 22 - protein, 27 aa, synthetic construct
  REGION 1..27: note = transportan
GWTLNSAGYLLGKINLKALAALAKKIL

SEQ ID NO: 23 - protein, 16 aa, synthetic construct
  REGION 1..16: note = p-Antp
RQIKIWFQNRRMKWKK

SEQ ID NO: 24 - protein, 15 aa, synthetic construct
  REGION 1..15: note = hCT(18-32)
KFHTFPQTAIGVGAP

SEQ ID NO: 25 - protein, 18 aa, synthetic construct
  REGION 1..18: note = KLA
KLALKLALKALKAALKLA

SEQ ID NO: 26 - protein, 8 aa, synthetic construct
  REGION 1..8: note = Oligoarginine
RRRRRRRR